(19) United States
(12) Patent Application Publication
(10) Pub. No.: US 2026/0021076 A1
(43) Pub. Date: Jan. 22, 2026

(54) ISOXAZOLINE AND MACROCYCLIC LACTONE MICROSPHERES

(71) Applicant: Zoetis Services LLC, Parsippany, NJ (US)

(72) Inventor: John Maxwell MAZZARA, Kalamazoo, MI (US)

(21) Appl. No.: 19/275,150

(22) Filed: Jul. 21, 2025

Related U.S. Application Data
(60) Provisional application No. 63/674,018, filed on Jul. 22, 2024.

(51) Int. Cl. A61K 31/422; A61K 9/16; A61K 31/365

(52) U.S. Cl. CPC A61K 31/422; A61K 9/1617; A61K 9/1623; A61K 9/1647; A61K 9/1652; A61K 31/365

(57) ABSTRACT

The invention describes an extended-release composition comprising PLGA or PLA polymeric microspheres comprising an isoxazoline, preferably sarolaner, and a macrocyclic lactone, preferably moxidectin, and wherein the composition further comprises at least one pharmaceutically acceptable excipient; and uses thereof.

### FIELD OF INVENTION

This invention relates to a novel extended-release injectable antiparasiticidal composition comprising polymeric microspheres containing both an isoxazoline and a macrocyclic lactone, as well as a method for treating an animal with a parasitic infection or infestation by administering the microsphere composition to an animal in need thereof.

### BACKGROUND OF THE INVENTION

The present invention relates to a novel extended-release injectable veterinary composition comprising polymeric microspheres containing both an isoxazoline compound and a macrocyclic lactone for treating an animal with a parasitic infestation. The polymeric composition is prepared as a suspension composition for subcutaneous injection. The present invention provides an improved injectable dual-active extended-release microsphere for the treatment of a parasitic infestation in an animal. The injectable polymeric composition has unique properties including: versatile drug release kinetics, duration of efficacy and established safety.

Polymeric microparticles and microspheres have been used for drug delivery for numerous pharmaceutical uses. For example, U.S. Pat. No., 6,340,671 describes extended-release microsphere (glyceryl tristearate and a neutral triglyceride oil) compositions of moxidextin; U.S. Pat. No., 6,726,918 describes a bio-degradable implant containing dexamethasone for treating inflammatory ocular conditions; and US Patent Application No., 2006-0173060 describes biopolymer microparticles containing an alpha-2-adrenergic receptor agonist for treating glaucoma. U.S. Pat. No. 6,340,671 describes stable moxidectin microspheres prepared with glyceryl tristearate in an injectable formulation. This tristearate formulation was commercialized as Proheart 6 (month) and 12 (month). The half-life for moxidectin was reported to be 73 days. Proheart 6 is a 0.17 mg/kg dose wherein Proheart 12 is a 0.5 mg/kg dose, accounting for a 3× increase in the mass/number of microspheres added to the composition. Even though the mass/number of microspheres increase 3×, the moxidectin concentration within each respective microsphere remains at 10%. The increased dose is achieved at an identical injection volume of 0.05 mL/kg body weight. WO2016-138339 describes the use of a block co-polymer of ethylene oxide and propylene oxide (poloxamer) to prepare microspheres containing isoxazolines. WO2021/233967 describes the use of polycaprolactone for preparing a microsphere with a macrocyclic lactone which is then admixed with isoxazoline particles to prepare an injectable composition.

Compositions currently available for parasitic treatment generally include topical, oral, and injectable compositions for animals and do not always demonstrate good activity, good speed of action, or a long duration of action. In particular, an advantageous injectable pharmaceutical composition for veterinary application is desirable, that enables a single injection to provide efficacious concentration levels of both classes of active compounds, an isoxazoline compound such as sarolaner as well as a macrocyclic lactone such as moxidectin, in blood plasma of the treated animals over an extended period.

Thus, there is a need for a stable, extended-release, easily administered, and effective extended-release injectable antiparasitic composition.

The veterinary biopolymeric composition of the present invention provides extended-release efficacy in animals against ectoparasites and endoparasites.

### SUMMARY OF THE INVENTION

The present invention relates to a novel extended-release antiparasitic polymeric microsphere composition, wherein the microsphere contains both an isoxazoline and a macrocyclic lactone, and the microsphere is prepared from a poly (D,L-lactide-co-glycolide) (PLGA) polymer or a poly (D,L-lactic acid) (PLA) polymer. The composition can be used for the prevention and treatment of a parasitic infection or infestation in an animal. Further, the invention contemplates the prevention of tick-borne diseases, for example, anaplasmosis, babesiosis, Lyme disease, theileriosis, ehrlichiosis, and the like. Thus, according to the present invention, there is provided an improved extended-release polymeric microsphere injectable composition.

In one aspect, the extended-release injectable composition comprises polymeric microspheres containing an isoxazoline and a macrocyclic lactone, and optionally, an antioxidant. In another aspect, the extended-release injectable composition comprises polymeric microspheres containing an isoxazoline and a macrocyclic lactone, and optionally, an antioxidant, and wherein the polymer is selected from the group consisting of poly (D,L-lactic acid) (PLA), polyglycolide (PGA), poly (D,L-lactide-co-glycolide) (PLGA), polycaprolactone (PCL), poly (D,L-lactide-co-caprolactone) (PLACL), poly (glycolide-co-caprolactone) (PGACL), glyceryl tristearate (GTS), poloxomer and any mixture thereof, and wherein the injectable composition further comprises at least one pharmaceutically acceptable excipient. The preferred microspheres comprise a PLGA or PLA polymer; more particularly, the PLGA polymer.

The polymeric microspheres of the invention contain a total combined drug load of about 54% to about 62%. Individual amounts of moxidectin range from about 5% to about 10% and individual amounts of sarolaner range from about 49% to about 54%. In another aspect, the polymeric microspheres contain about 6.5% to about 10% moxidectin and about 49% to about 52% sarolaner. In another aspect, the polymeric microspheres contain about 8% to about 10% moxidectin and about 49% to about 52% sarolaner. In one aspect, the polymeric microsphere contains about 8% moxidectin and about 49% to about 52% sarolaner. In another aspect, the polymeric microsphere contains about 9% moxidectin and about 49% to about 52% sarolaner. In one aspect, the polymerc microsphere contains 8% moxidectin and 52% sarolaner for a moxidectin-to-sarolaner ratio of 0.77:5 (i.e., 8/52=0.154; 0.154:1 or 0.77:5). In another aspect, the polymeric microsphere contains 9% moxidectin and 50% sarolaner for a moxidectin-to-sarolaner ratio of 0.9:5 (i.e., 9/50=0.180; 0.180:1 or 0.9:5). In another aspect, the polymeric microsphere contains 10% moxidectin and 49% sarolaner for a moxidectin-to-sarolaner ratio of 1.02:5 (i.e., 10/49=0.204; 0.204:1 or 1.02:5˜1:5). In each of these polymeric microspheres, the polymer is a PLGA polymer or a PLA polymer, preferably a PLGA polymer.

In another aspect, the extended-release injectable composition comprises polymeric microspheres containing an isoxazoline and a macrocyclic lactone, and optionally, an antioxidant, and wherein the polymer is PLGA, and wherein the injectable composition further comprises at least one pharmaceutically acceptable excipient. In another aspect, the extended-release injectable composition comprises polymeric microspheres containing an isoxazoline, a macrocyclic lactone and an antioxidant, and wherein the polymer is PLGA, and wherein the injectable composition further comprises at least one pharmaceutically acceptable excipient. In another aspect, the extended-release injectable composition comprises polymeric microspheres containing sarolaner, moxidectin and optionally an antioxidant, and wherein the polymer is PLGA, and wherein the injectable composition further comprises at least one pharmaceutically acceptable excipient. In another aspect, the extended-release injectable composition comprises polymeric microspheres containing sarolaner, moxidectin and an antioxidant, and wherein the polymer is PLGA, and wherein the injectable composition further comprises at least one pharmaceutically acceptable excipient. In another aspect, the extended-release injectable composition comprises polymeric microspheres containing sarolaner, moxidectin, an antioxidant and mannitol, and wherein the polymer is PLGA, and wherein the PLGA is 50% poly (D, L-lactide) and 50% polyglycolide or the polymer is 75% poly (D,L-lactide) and 25% polyglycolide, and wherein the injectable composition further comprises at least one pharmaceutically acceptable excipient. In another aspect, the extended-release injectable composition comprises polymeric microspheres containing sarolaner, moxidectin and butylated hydroxytoluene, and wherein the polymer is PLGA, and wherein the PLGA is 75% poly (D, L-lactide) and 25% polyglycolide, and wherein the injectable composition further comprises at least one pharmaceutically acceptable excipient. In another aspect, the extended-release injectable composition comprises polymeric microspheres containing sarolaner, moxidectin and an antioxidant, and wherein the polymer is PLGA, and wherein the PLGA is 75% poly (D,L-lactide) and 25% polyglycolide, and wherein the injectable composition further comprises at least one pharmaceutically acceptable excipient selected from the group consisting of ethanol, propylene glycol, polyethylene glycol, 2-pyrrolidone, N-methylpyrrolidinone, polysorbate 20, mannitol, sodium carboxymethyl cellulose, water, triacetin and mixtures thereof. In another aspect, the extended-release injectable composition comprises polymeric microspheres containing sarolaner, moxidectin and an antioxidant, and wherein the polymer is PLGA, and wherein the PLGA is 75% poly (D,L-lactide) and 25% polyglycolide, and wherein the injectable composition further comprises at least one pharmaceutically acceptable excipient selected from the group consisting of polysorbate 20, mannitol, sodium carboxymethyl cellulose, water and mixtures thereof; and optionally an antioxidant. In another aspect, the extended-release injectable composition comprises polymeric microspheres containing sarolaner, moxidectin and an antioxidant, and wherein the polymer is PLGA, and wherein the PLGA is 75% poly (D,L-lactide) and 25% polyglycolide, and wherein the injectable composition further comprises at least one pharmaceutically acceptable excipient selected from the group consisting of mannitol, sodium carboxymethyl cellulose, polysorbate 20, water, butylated hydroxytoluene and mixtures thereof.

In another aspect, the extended-release injectable composition comprises polymeric microspheres containing sarolaner, moxidectin and butylated hydroxytoluene, and wherein the polymer is PLGA, and wherein the PLGA is 75% poly (D, L-lactide) and 25% polyglycolide with an inherent viscosity of about 0.26 to 0.43 dL/g; and wherein the total combined drug load is about 54% to about 62% with a moxidectin: sarolaner ratio of about 0.50-1:5, and the particle size of the microsphere is about 20 μm to about 40 μm; and wherein the injectable composition further comprises at least one pharmaceutically acceptable excipient. In another aspect, the extended-release injectable composition comprises polymeric microspheres containing sarolaner, moxidectin and butylated hydroxytoluene, and wherein the polymer is PLGA, and wherein the

PLGA is 75% poly (D,L-lactide) and 25% polyglycolide with an inherent viscosity of about 0.28 to 0.43 dL/g; and wherein the total combined drug load is about 54% to about 62% with a moxidectin: sarolaner ratio of about 0.63-1:5, and the particle size of the microsphere is about 24 μm to about 40 μm; and wherein the injectable composition further comprises at least one pharmaceutically acceptable excipient. In another aspect, the extended-release injectable composition comprises polymeric microspheres containing sarolaner, moxidectin and butylated hydroxytoluene, and wherein the polymer is PLGA, and wherein the PLGA is 75% poly (D,L-lactide) and 25% polyglycolide with an inherent viscosity of about 0.28 to 0.43 dL/g; and wherein the total combined drug load is about 57% to about 62% with a moxidectin: sarolaner ratio of about 0.63-1:5, and the particle size of the microsphere is about 24 μm to about 36 μm; and wherein the injectable composition further comprises at least one pharmaceutically acceptable excipient. In another aspect, the extended-release injectable composition comprises polymeric microspheres containing sarolaner, moxidectin and butylated hydroxytoluene, and wherein the polymer is PLGA, and wherein the PLGA is 75% poly (D, L-lactide) and 25% polyglycolide with an inherent viscosity of about 0.28 to 0.41 dL/g; and wherein the total combined drug load is about 57% to about 62% with a moxidectin: sarolaner ratio of about 0.63-1:5, and the particle size of the microsphere is about 24 μm to about 36 μm; and wherein the injectable composition further comprises at least one pharmaceutically acceptable excipient. In another aspect, the extended-release injectable composition comprises polymeric microspheres containing sarolaner, moxidectin and butylated hydroxytoluene, and wherein the polymer is PLGA, and wherein the PLGA is 75% poly (D,L-lactide) and 25% polyglycolide with an inherent viscosity of about 0.30 to 0.40 dL/g; and wherein the total combined drug load is about 57% to about 62% with a moxidectin: sarolaner ratio of about 0.75-1:5, and the particle size of the microsphere is about 25 μm to about 35 μm; and wherein the injectable composition further comprises at least one pharmaceutically acceptable excipient. In another aspect, the extended-release injectable composition comprises polymeric microspheres containing sarolaner, moxidectin and butylated hydroxytoluene, and wherein the polymer is PLGA, and wherein the PLGA is 75% poly (D,L-lactide) and 25% polyglycolide with an inherent viscosity of about 0.31 to 0.39 dL/g; and wherein the total combined drug load is about 57% to about 62% with a moxidectin: sarolaner ratio of about 0.75-1:5, and the particle size of the microsphere is about 26 μm to about 34 μm; and wherein the injectable composition further comprises at least one pharmaceutically acceptable excipient. In another aspect, the extended-release injectable composition comprises polymeric microspheres containing sarolaner, moxidectin and butylated hydroxytoluene, and wherein the polymer is PLGA, and wherein the PLGA is 75% poly (D,L-lactide) and 25% polyglycolide with an inherent viscosity of about 0.31 to 0.39 dL/g; and wherein the total combined drug load is about 57% to about 62% with a moxidectin: sarolaner ratio of about 0.75-1:5, and the particle size of the microsphere is about 26 μm to about 34 μm; and wherein the injectable composition further comprises at least one pharmaceutically acceptable excipient selected from the group consisting of mannitol, polysorbate 20, sodium carboxymethyl cellulose, water and mixtures thereof; and optionally an antioxidant. In another aspect, the extended-release injectable composition comprises polymeric microspheres containing sarolaner, moxidectin and butylated hydroxytoluene, and wherein the polymer is PLGA, and wherein the PLGA is 75% poly (D, L-lactide) and 25% polyglycolide with an inherent viscosity of about 0.31 to 0.39 dL/g; and wherein the total combined drug load is about 57% to about 62% with a moxidectin: sarolaner ratio of about 0.75-1:5, and the particle size of the microsphere is about 26 μm to about 34 μm; and wherein the injectable composition further comprises at least one pharmaceutically acceptable excipient selected from the group consisting of mannitol, polysorbate 20, sodium carboxymethyl cellulose, water and mixtures thereof; and further comprises the antioxidant butylated hydroxytoluene. In another aspect, the extended-release injectable composition comprises polymeric microspheres containing sarolaner, moxidectin and butylated hydroxytoluene, and wherein the polymer is PLGA, and wherein the PLGA is 75% poly (D,L-lactide) and 25% polyglycolide with an inherent viscosity of about 0.31 to 0.39 dL/g; and wherein the total combined drug load is about 58% to about 60% with a moxidectin: sarolaner ratio of about 0.75-1:5, and the particle size of the microsphere is about 26 μm to about 32 μm; and wherein the injectable composition further comprises at least one pharmaceutically acceptable excipient selected from the group consisting of mannitol, polysorbate 20, sodium carboxymethyl cellulose, water and mixtures thereof; and optionally an antioxidant. In another aspect, the extended-release injectable composition comprises polymeric microspheres containing sarolaner, moxidectin and butylated hydroxytoluene, and wherein the polymer is PLGA, and wherein the PLGA is 75% poly (D,L-lactide) and 25% polyglycolide with an inherent viscosity of about 0.31 to 0.39 dL/g; and wherein the total combined drug load is about 58% to about 60% with a moxidectin: sarolaner ratio of about 0.75-1:5, and the particle size of the microsphere is about 26 μm to about 32 μm; and wherein the injectable composition further comprises at least one pharmaceutically acceptable excipient selected from the group consisting of mannitol, polysorbate 20, sodium carboxymethyl cellulose, water and mixtures thereof; and further comprises the antioxidant butylated hydroxytoluene. In another aspect, the extended-release injectable composition comprises polymeric microspheres containing sarolaner, moxidectin and optionally, butylated hydroxytoluene, and wherein the polymer is PLGA, and wherein the PLGA is 75% poly (D, L-lactide) and 25% polyglycolide with an inherent viscosity of about 0.39 dL/g; and wherein the total combined drug load is about 59% with a moxidectin: sarolaner ratio of about 0.75-1:5, and the particle size of the microsphere is about 26 μm to about 32 μm; and wherein the injectable composition further comprises at least one pharmaceutically acceptable excipient selected from the group consisting of mannitol, polysorbate 20, sodium carboxymethyl cellulose, water and mixtures thereof; and further comprises the antioxidant butylated hydroxytoluene. In another aspect, the extended-release injectable composition comprises polymeric microspheres containing sarolaner, moxidectin and butylated hydroxytoluene, and wherein the polymer is PLGA, and wherein the PLGA is 75% poly (D,L-lactide) and 25% polyglycolide with an inherent viscosity of about 0.39 dL/g; and wherein the total combined drug load is about 59% with a moxidectin: sarolaner ratio of about 0.96-1:5, and the particle size of the microsphere is about 26 μm to about 32 μm; and wherein the injectable composition further comprises at least one pharmaceutically acceptable excipient selected from the group consisting of mannitol, polysorbate 20, sodium carboxymethyl cellulose, water and mixtures thereof; and further comprises the antioxidant butylated hydroxytoluene.

In another aspect, the extended-release injectable composition comprises polymeric microspheres containing sarolaner, moxidectin and an antioxidant, and wherein the polymer is PLA (100%), and wherein the injectable composition further comprises at least one pharmaceutically acceptable excipient selected from the group consisting of ethanol, propylene glycol, polyethylene glycol, 2-pyrrolidone, N-methylpyrrolidinone, polysorbate 20, mannitol, sodium carboxymethyl cellulose, water, triacetin and mixtures thereof. In another aspect, the extended-release injectable composition comprises polymeric microspheres containing sarolaner, moxidectin and an antioxidant, and wherein the polymer is PLA (100%), and wherein the injectable composition further comprises at least one pharmaceutically acceptable excipient selected from the group consisting of polysorbate 20, mannitol, sodium carboxymethyl cellulose, water and mixtures thereof. In another aspect, the extended-release injectable composition comprises polymeric microspheres containing sarolaner, moxidectin and an antioxidant, and wherein the polymer is PLA (100%), and wherein the injectable composition further comprises at least one pharmaceutically acceptable excipient selected from the group consisting of mannitol, sodium carboxymethyl cellulose, polysorbate 20, water and mixtures thereof. In another aspect, the extended-release injectable composition comprises polymeric microspheres containing sarolaner, moxidectin and an antioxidant, and wherein the polymer is PLA (100%), and wherein the injectable composition further comprises at least one pharmaceutically acceptable excipient which is a mixture of mannitol and sodium carboxymethyl cellulose. In another aspect, the extended-release injectable composition comprises polymeric microspheres containing sarolaner, moxidectin, and an antioxidant, and wherein the polymer is PLA (100%) with an inherent viscosity of about 0.6 to about 1.0 dL/g; and wherein the total combined drug load is about 50% to about 65%, or 54% to about 65%; or about 54% to about 60%, with a moxidectin: sarolaner ratio of about 0.6-1:5; the particle size of the micrsophere is about 20 μm to about 40 μm (preferably about 25 μm to about 38 μm); and wherein the injectable composition further comprises at least one pharmaceutically acceptable excipient. In another aspect, the extended-release injectable composition comprises polymeric microspheres containing sarolaner, moxidectin, and butylated hydroxytoluene, and wherein the polymer is PLA (100%) with an inherent viscosity of about 0.6 to about 1.0 dL/g; and wherein the total combined drug load is about 50% to about 65%, preferably about 54% to about 65%, with a moxidectin: sarolaner ratio of about 0.6-1:5; the particle size of the micrsophere is about 20 μm to about 40 μm (preferably about 255 μm to about 38 μm); and wherein the injectable composition further comprises at least one pharmaceutically acceptable excipient selected from the group consisting of mannitol, polysorbate 20, sodium carboxymethyl cellulose, water and mixtures thereof; and optionally an antioxidant. In another aspect, the extended-release injectable composition comprises polymeric microspheres containing sarolaner, moxidectin, and butylated hydroxytoluene, and wherein the polymer is PLA (100%) with an inherent viscosity of about 0.6 to about 1.0 dL/g; and wherein the total combined drug load is about 50% to about 65% or 54% to about 65% or about 54% to about 60%; with a moxidectin: sarolaner ratio of about 0.6-1:5; the particle size of the micrsophere is about 20 μm to about 40 μm (preferably about 25 μm to about 38 μm); and wherein the injectable composition further comprises at least one pharmaceutically acceptable excipient selected from the group consisting of mannitol, polysorbate 20, sodium carboxymethyl cellulose, water, an antioxidant and mixtures thereof. The preferred compositional antioxidant is butylated hydroxytoluene or butylated hydroxyanisole; and more preferably hydroxy butylatedtoluene.

In some instances, the macrocyclic lactone, particularly moxidectin, can be obtained already pre-mixed with butylated hydroxytoluene or butylated hydroxyanisole. In some instances, the macrocyclic lactone, moxidectin is obtained already pre-mixed with butylated hydroxytoluene. The polymeric microspheres of the invention comprise moxidectin and butylated hydroxytoluene as well as an isoxazoline, preferably sarolaner.

In another aspect, is a method for treating a parasitic infestation in an animal comprising administering the extended-release injectable polymeric microsphere composition to the animal in need thereof. In another aspect, the extended-release injectable polymeric microsphere composition is administered to the animal by subcutaneous injection once every 6-months or 12 months. In another aspect, the PLGA polymer composition can be used once every 6-months. In another aspect, the PLA composition can be used once every 12 months. In another aspect, the extended-release injectable PLGA polymeric microsphere composition is administered to the animal by subcutaneous injection once every 6-months. In another aspect, the extended-release injectable polymeric microsphere PLA composition is administered to the animal by subcutaneous injection once every 12 months. In another aspect, the animal is a companion animal. In another aspect, the companion animal is canine and feline. In another aspect, the companion animal is canine. In another aspect, the companion animal is feline.

In another aspect, is the use of the extended-release injectable polymeric microsphere composition to prepare a medicament for treating a parasitic infestation in an animal. In another aspect, the extended-release injectable polymeric microsphere medicament is administered by subcutaneous injection once every 6-months. In another aspect, the animal is a companion animal. In another aspect, the companion animal is canine and feline. In another aspect, the companion animal is canine. In another aspect, the animal is feline.

In another aspect, the extended-release injectable polymeric microsphere composition comprises sarolaner and moxidectin, and optionally butyl hydroxytoluene, that can be administered with at least one additional antiparasitic agent. The additional antiparasitic agent can be co-administered with the extended-release polymeric microsphere composition of the invention or the additional antiparasistic agent can be administered on a monthly basis, or as needed, per labeling guidelines. The additional antiparasitic agents include, for example, monepantel, thiabendazole, fenbendazole, oxfendazole, albendazole, paraherquamide, praziquantel, octadepsipeptide, oxantel, epsiprantel, levamisole, morantel, pyrantel (pamoate =embonate), febantel and spinosad.

In yet another aspect of the invention, is a process for preparing the polymeric microspheres by dissolving the isoxazoline and the macrocyclic lactone (optionally premixed with an antioxidant), in a solvent, preferably dichloromethane, and then mixing the drug solution in a homogenizer with an aqueous solution containing polyvinyl alcohol to prepare the microspheres. The microspheres are collected and admixed with at least one pharmaceutically acceptable excipient, preferably mannitol and optionally with sodium carboxymethyl cellulose and/or polysorbate 20. The admixed microspheres are bottled and lyophilized. The lyophilized microspheres are later resuspended when needed for administration by adding a diluent to the microspheres. The diluent comprises at least water, and optionally sodium carboxymethyl cellulose and/or polysorbate 20. After resuspension of the lyophilized microspheres in the diluent, the suspension can then be administered to an animal in need thereof, by subcutaneous injection, for the prevention and/or treatment of a parasitic infection or infestation.

### Definitions

For purposes of the present invention, as described and claimed herein, the following terms and phrases are defined as follows:

“About” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater.

“Animal” as used herein, unless otherwise indicated, refers to an individual animal, and said individual animal is a mammal. Specifically, mammal refers to a vertebrate animal that is human and non-human, which are members of the taxonomic class Mammalia. Non-exclusive examples of non-human animals include companion animals and livestock. Non-exclusive examples of a companion animal include: dog (canine), cat (feline) and horse (equine). Preferred companion animals are dog and cat.

“Drug Load”, as used herein, unless otherwise indicated, refers to the amount (%) of isoxazoline (e.g., sarolaner) and macrocyclic lactone (e.g., moxidectin) in the microspheres of the extended-release composition.

“Excipient”, as used herein, unless otherwise indicated, refers to pharmaceutically acceptable excipients that are included in the injectable microsphere composition including, for example, viscosity and isotonic modifiers, binders, surfactants, preservatives, antioxidants, dispersants and the like.

“Infection”, as used herein, unless otherwise indicated, refers to the state or condition of having parasites in and/or on the body. Furthermore, the infection may lead to an infestation on or in the animal.

“Extended-release”, as used herein, unless otherwise indicated, refers to the duration of time between dosing. The duration refers to administration of the polymeric microsphere composition at least once every 6-months or at least once every 12 months.

“Microsphere”, as used herein, unless otherwise indicated, refers to a solid, semi-solid or amorphous sphere formed from a polymer having an isoxazoline and macrocyclic lactone dispersed throughout, and optionally, an antioxidant.

“Parasite(s)”, as used herein, unless otherwise indicated, refers to ectoparasites and endoparasites. Ectoparasites are organisms which feed through or upon the skin of its host. Endoparasites include those parasites that live within the tissues and organs of their host.

“Pharmaceutically acceptable” as used herein, unless otherwise indicated, suggests that the substance or composition must be compatible chemically and/or toxicologically with the other ingredients comprising the composition and/or the animal being treated therewith.

“Polymer” as used herein, unless otherwise indicated, refers to a biodegradable, bioerodable and/or biocompatible polymer. A biocompatible polymer refers to a polymer that when administered to an animal does not induce a significant inflammatory response. A biodegradable (bioerodable) polymer is a polymer that degrades in vivo, and wherein erosion of the polymer or polymers over time occurs concurrent with or subsequent to release of the therapeutic agent (i.e., an isoxazoline and macrocyclic lactone). Specifically, hydrogels such as methylcellulose which act to release drug through polymer swelling are specifically excluded from the term polymer. A polymer may be a homopolymer, a copolymer or a polymer comprising more than two different polymeric units. A polymeric composition, as described herein, refers to a composition comprising at least one polymer; particularly a PLGA or PLA polymer, and more particularly, a PLGA polymer.

“Ratio”, as used herein, unless otherwise indicated, refers to the amount (%) of drug (macrocylic lactone and isoxazoline) relative to each other in the microsphere. The ratio is based on the % of moxidectin and the % of sarolaner in the microsphere and is represented as moxidectin: sarolaner (e.g., 1:5 which refers to 10% moxidectin and 50% sarolaner).

“Therapeutically effective amount”, as used herein, unless otherwise indicated, refers to an amount of an isoxazoline and macrocyclic lactone that (i) treats or prevents the particular parasitic infestation, (ii) attenuates, ameliorates, or eliminates one or more symptoms of the particular parasitic infestation, or (iii) prevents or delays the onset of one or more symptoms of the particular parasitic infestation described herein. A dose range of about 4-15 mg/kg sarolaner and 0.4-1 mg/kg moxidectin is contemplated to be a therapeutically effective dose. For a 6-month duration injection, a dose range of about 5 mg/kg sarolaner and 0.6-1 mg/kg moxidectin is contemplated to be a therapeutically effective dose. For a 12-month injection, a dose range of about 10 mg/kg sarolaner and 1.2-2 mg/kg moxidectin is contemplated to be a therapeutically effective dose.

“Treatment”, “treating”, and the like, as used herein, unless otherwise indicated, refers to reversing, alleviating, or inhibiting the parasitic infestation. As used herein, these terms also encompass, depending on the condition of the animal preventing the onset of a disorder or condition, or of symptoms associated with a disorder or condition, including reducing the severity of a disorder or condition or symptoms associated therewith prior to affliction with said infestation. Thus, treatment can refer to administration of the composition of the present invention to an animal that is not at the time of administration afflicted with the parasitic infestation, for example, as prophylactic treatment. Treating also encompasses preventing the recurrence of an infestation or of symptoms associated therewith as well as references to “control” (e.g., kill, repel, expel, incapacitate, deter, eliminate, alleviate, minimize, and eradicate).

The phrase, “at least one”, as used herein, unless otherwise noted, refers to one or more when referring to a pharmaceutically acceptable excipient(s) and/or additional antiparasitic agent(s). At least one can refer to water and it can also refer to water, mannitol and polysorbate 20.

### DETAILED DESCRIPTION

### Figure Description

FIG. 1. Moxidectin Plasma Concentrations For Different Combined Drug Loaded Microspheres and Microsphere Particle Sizes

FIG. 2a. Moxidectin and Sarolaner Microsphere Pharmacokinetics with a 51.7% Combined Drug Load

FIG. 2b. Moxidectin and Sarolaner Microsphere Pharmacokinetics with a 59.4% Combined Drug Load

FIG. 3. 56% Combined Drug Loaded Microspheres with 2:5 Ratio of Moxidectin: Sarolaner

FIG. 4. 56% Combined Drug Loaded Microspheres with 3:5 Ratio of Moxidectin: Sarolaner

FIG. 5. Moxidectin Plasma Concentrations for the PLGA (75:25) and Glyceryl Tristearate Microsopheres

FIG. 6a: Moxidectin Plasma Concentrations for the PLGA Microspheres with Constant Inherent Viscosity (0.39 dL/g) and Particle Size (30-34 μm) with Varying Drug Load Amounts and Moxidectin: Sarolaner Ratios

FIG. 6b: Sarolaner Plasma Concentrations for the PLGA Microspheres with Constant Inherent Viscosity (0.39 dL/g) and Particle Size (30-34 μm) with Varying Drug Load Amounts and Moxidectin: Sarolaner Ratios

FIG. 7a: Moxidectin Plasma Concentrations for the PLGA Microspheres with Constant Total Drug Load (59-60%) and Moxidectin: Sarolaner Ratio (0.76:5) with Varying Particle Size and Polymer Inherent Viscosity

FIG. 7b: Sarolaner Plasma Concentrations for the PLGA Microspheres with Constant Total Drug Load (59-60%) and Moxidectin: Sarolaner Ratio (0.76:5) with Varying Particle Size and Polymer Inherent Viscosity

It is to be understood by one of ordinary skill in the art that the present discussion is a description of exemplary embodiments only and is not intended as limiting the broader aspects of the present invention, which broader aspects are embodied in the exemplary construction. In fact, it will be apparent to those skilled in the art that various modifications and variations can be made in the present invention without departing from the scope or spirit of the invention. For instance, features illustrated or described as part of one embodiment can be used in another embodiment to yield a still further embodiment. It is intended that the present invention cover such modifications and variations as come within the scope of the appended claims and their equivalents.

The isoxazoline compounds that can be incorporated into the microspheres of the invention include isoxazolines as described in WO2007/079162, WO2008/154528, WO2009/002809, WO2011/149749, WO2013/078070, WO2014/439475, U.S. Pat. No. 8,466,115, WO2012/120399, WO2012/120135, WO2014/039484 and WO2014/189837. In particular, the isoxazoline that can be incorporated into the microsphere with a macrocyclic lactone is selected from the group consisting of sarolaner ((S)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethan-1-one), afoxoalaner ((4-(5-(3-chloro-5-(trifluoromethyl)phenyl)-5-(trifluoromethyl)-4,5-dihydroisoxazol-3-yl)-N-(2-oxo-2-((2,2,2-trifluoroethyl)amino)ethyl)-1-naphthamide)), fluralaner (4-(5-(3,5-dichlorophenyl)-5-(trifluoromethyl)-4,5-dihydroisoxazol-3-yl)-2-methyl-N-(2-oxo-2-((2,2,2-trifluoroethyl)amino)ethyl)benzamide), lotilaner (3-methyl-N-(2-oxo-2-((2,2,2-trifluoroethyl)amino)ethyl)-5-(5-(3,4,5-trichlorophenyl)-5-(trifluoromethyl)-4,5-dihydroisoxazol-3-yl)thiophene-2-carboxamide), mivorilaner ((S)-3-(5-(3,5-dichloro-4-fluorophenyl)-5-(trifluoromethyl)-4,5-dihydroisoxazol-3-yl)-N-(2-((2,2-difluoroethyl)amino)-2-oxoethyl)-5,6-dihydro-4H-cyclopenta[c]thiophene-1-carboxamide) and umifoxolaner (4-(5-(3-chloro-4-fluoro-5-(trifluoromethyl)phenyl)-5-(trifluoromethyl)-4,5-dihydroisoxazol-3-yl)-N-(2-oxo-2-((2,2,2-trifluoroethyl)amino)ethyl)-1-naphthamide), and stereoisomers thereof. Esafoxolaner is the S enantiomer of afoxolaner. The preferred isoxazoline is sarolaner. Structures of these additional isoxazolines are depicted below:

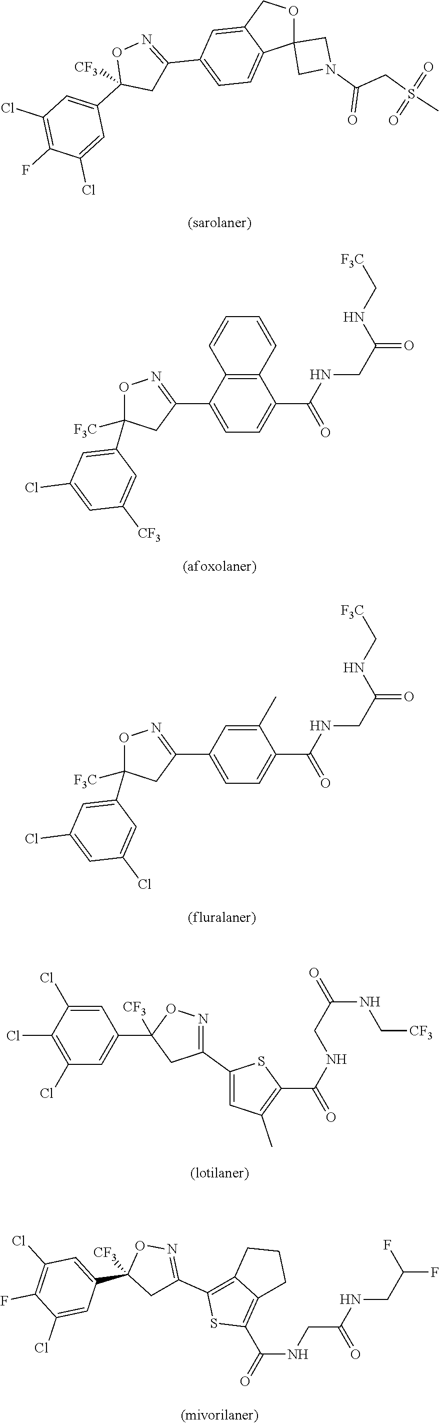 [SMILES: CS(=O)(=O)CC(=O)N1CC2(C1)OCc1cc(C3=NO[C@@](c4cc(Cl)c(F)c(Cl)c4)(C(F)(F)F)C3)ccc12 ; O=C(CNC(=O)c1ccc(C2=NOC(c3cc(Cl)cc(C(F)(F)F)c3)(C(F)(F)F)C2)c2ccccc12)NCC(F)(F)F ; Cc1cc(C2=NOC(c3cc(Cl)cc(Cl)c3)(C(F)(F)F)C2)ccc1C(=O)NCC(=O)NCC(F)(F)F ; Cc1cc(C2=NOC(c3cc(Cl)c(Cl)c(Cl)c3)(C(F)(F)F)C2)sc1C(=O)NCC(=O)NCC(F)(F)F ; O=C(CNC(=O)c1sc(C2=NO[C@@](c3cc(Cl)c(F)c(Cl)c3)(C(F)(F)F)C2)c2c1CCC2)NCC(F)F]

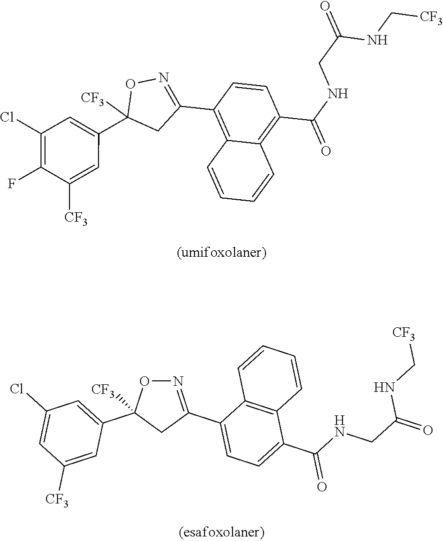 [SMILES: O=C(CNC(=O)c1ccc(C2=NOC(c3cc(Cl)c(F)c(C(F)(F)F)c3)(C(F)(F)F)C2)c2ccccc12)NCC(F)(F)F ; O=C(CNC(=O)c1ccc(C2=NO[C@@](c3cc(Cl)cc(C(F)(F)F)c3)(C(F)(F)F)C2)c2ccccc12)NCC(F)(F)F]

The macrocyclic lactones, include abamectin, doramectin, emamectin, eprinomectin, ivermectin, milbemcycin, milbemycin oxime, moxidectin, selamectin, and the like. The preferred macrocyclic lactone is moxidectin.

The present invention provides for a extended-release polymeric microsphere composition for the treatment of a parasitic infestation in an animal wherein the microsphere comprises a therapeutically effective amount of an isoxazoline and a macrocyclic lactone, and optionally, an antioxidant.

Veterinary compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

In the present invention, the injectable composition is a suspension composition comprising polymeric microspheres. Polymers can be either a biodegradable (or bioerodable) polymer or a biocompatible polymer, or a mixture thereof. “Polymer” or “polymeric”, as used herein, further refers to oligomers, adducts, homopolymers, random copolymers, pseudo-copolymers, statistical copolymers, alternating copolymers, periodic copolymer, bipolymers, terpolymers, quaterpolymers, other forms of copolymers, substituted derivatives thereof, and combinations of two or more thereof (i.e., polymer blends). The polymers can be linear, branched, block, graft, monodisperse, polydisperse, regular, irregular, tactic, isotactic, syndiotactic, stereoregular, atactic, stereoblock, single-strand, double-strand, star, comb, dendritic, and/or ionomeric. Combinations of polymers can be used if biocompatible. Non-limiting examples of polymers include: polylactide, polyglycolide, and poly (lactid-co-glycolide) copolymers. Further, non-limiting examples of polymers include: poly (D,L-lactide-co-glycolide) (PLGA), poly (D,L-lactide) (PLA), poly (L-lactide) (PLLA), polyglycolide (PGA), polycaprolactone (PCL), poly (D, L-lactide-co-caprolactone) (PLACL), poly (glycolide-co-caprolactone) (PGACL), poly (D,L-lactide-co-glycolide-co-caprolactone) (PLGACL), glyceryl tristearate (GTS), polyethylene glycol (PEG), polydioxanone (PDO), poly (hydroxyl alkanoate) (PHA), poly (vinyl alcohol) (PVA), or other polyesters (e.g., poly (orthoesters), poly (aminoesters), poly (anhydrides), poly (D,L-lactide-caprolactone), poloxamers, poly (organophosphazenes)), or any combination thereof. PLGA herein refers to the synonymous terms: poly (D,L-lactide-co-glycolide; poly (D,L-lactide-co-glycolic acid) and poly (D,L-lactic acid-co-glycolic acid).

The polymers polylactic acid, polyglycolic acid, and polylactic-glycolic acid copolymer (PLGA), have been investigated extensively. These polymers are polyesters that, upon administration to an animal, undergo simple hydrolysis of its ester linkages. The products of such hydrolysis are biologically compatible and metabolizable moieties (e.g., lactic acid and glycolic acid), which are eventually removed from the body through normal physiological processes.

Polymer biodegradation products are formed at a very slow rate, and hence do not affect normal cell function. To minimize the toxicity of the intact polymer carrier and its degradation products, polymers have been designed based on naturally occurring metabolites. The preferred polymer is a poly (D, L-lactide-co-glycolide) comprising a mixture of polyglycolide (PGA) and poly (D,L-lactide) (PLA). In various instances in which the polymer is poly (D,L-lactide-co-glycolide), there is 95% poly (D,L-lactide) and 5% polyglycolide; 90% poly (D,L-lactide) and 10% polyglycolide; 85% poly (D,L-lactide) and 15% polyglycolide; 80% poly (D,L-lactide) and 20% polyglycolide; 75% poly (D,L-lactide) and 25% polyglycolide; 70% poly (D,L-lactide) and 30% polyglycolide; 65% poly (D,L-lactide) and 35% polyglycolide; 60% poly (D,L-lactide) and 40% polyglycolide; 55% poly (D,L-lactide) and 45% polyglycolide; 50% poly (D,L-lactide) and 50% polyglycolide; 45% poly (D,L-lactide) and 55% polyglycolide; 40% poly (D,L-lactide) and 60% polyglycolide; 35% poly (D,L-lactide) and 65% polyglycolide; 30% poly (D,L-lactide) and 70% polyglycolide; 25% poly (D,L-lactide) and 75% polyglycolide; 20% poly (D,L-lactide) and 80% polyglycolide; 15% poly (D,L-lactide) and 85% polyglycolide; 10% poly (D,L-lactide) and 90% polyglycolide; and 5% poly (D, L-lactide) and 95% polyglycolide. A preferred PLA: PGA ratio is about 50:50 or 75:25. The more preferred PLA: PGA (L: G) ratio is about 75:25. A preferred PLGA is the RESOMER® polymers which are bioresorbable aliphatic polyesters comprised of a range of different ratios of lactide and glycolide monomers and PLA stereochemistries with acidic end-capped functionalizations. PLGA's can also have ester end capped functionalizations which tend to make the polymer more resistant to hydrolytic degradation. The preferred 75:25 lactide/glycolide polymer provides a depot with drug release for at least 6 months and the PLA polymer provides a depot with drug release for at least 6 to 12 months. Other polymers that can be used in preparing the dual-active microspheres include, for example, poly (ethylene oxide), poly (ethylene glycol), polyvinylpyrrolidones, polycaprolactones and the like.

Inherent viscosity (IV) is a viscometric method for measuring molecular size by comparing the flow time of a polymer solution through a capillary to the flow time of a pure solvent through the same capillary and is calculated as the ratio of the natural logarithm of a polymers relative viscosity to its mass concentration. The units of IV are typically reported in deciliers per gram (dL/g). As the IV value increases, there is a correlative increase in polymer molecular weight. The IV of the polymer is also an important characteristic in controlling the biodegradation of the polymer. For the dual active microspheres, the preferred IV of the PLGA polymer ranges from about 0.26 to about 0.43 dL/g, about 0.28 to about 0.43 dL/g, about 0.28 to about 0.41 dL/g, about 0.30 to about 0.40 dL/g, or about 0.31 to about 0.39 dL/g. A preferred IV is about 0.31 to about 0.39 dL/g. A preferred IV is about 0.31 dL/g, about 0.32 dL/g, about 0.33 dL/g, about 0.34 dL/g, about 0.35 dL/g, about 0.36 dL/g, about 0.37 dL/g, about 0.38 dL/g or about 0.39 dL/g. For the PLA polymer, IV ranges from about 0.6 to 1.0 dL/g.

The release of the isoxazoline and macrocyclic lactone from these polymeric systems can occur by two different mechanisms. The active agent can be released by diffusion from the surface of the microspheres or through aqueous filled channels generated in the dosage form by the dissolution of the active agent or by voids created by the removal of the polymer solvent during the original microencapsulation. The second mechanism is enhanced release due to the degradation and erosion of the polymer. With time, the polymer begins to erode and generates increased porosity and microstructure within the device. This creates additional pathways for drug release. The degradation of the polymers occurs by spontaneous hydrolysis of the ester linkages on the backbone. Thus, the rate can be controlled by changing polymer properties influencing water uptake. These include the monomer ratio (lactide to glycolide), the use of L-lactide as opposed to D,L lactide, drug loading and the polymer molecular weight. These factors determine the hydrophilicity and crystallinity which ultimately govern the rate of water penetration. Hydrophilic components such as salts, carbohydrates and surfactants can also be incorporated to increase water penetration into the devices and thus accelerate the erosion of the polymer, these hydrophilic components are also construed as excipients. By altering the properties of the polymer and the properties of the dosage form, one can control the contribution of each of these release mechanisms and alter the release rate of active agent. Slowly eroding polymers such as poly L-lactide or high molecular weight poly (lactide-co-glycolide) with low glycolide compositions will cause the release to become diffusion controlled. Increasing the glycolide composition and decreasing the molecular weight enhances both water uptake and the hydrolysis of the polymer and adds an erosion component to the release kinetics. The release rate can also be controlled by varying the loading of active agent within the microspheres. Increasing the drug load will increase the network of interconnecting channels formed upon the dissolution of the active agent and enhance the release of drug from the microspheres.

As described above, release of the active agents from the polymer can be controlled, in part, by the polymeric composition. Various factors such as the mechanical strength, swelling behavior, capacity to undergo hydrolysis all can affect release rates of the polymer. The polymer can be engineered and specifically designed and/or selected to provide the polymeric composition with the desired biodegradation rate and release profile of the active agent for a selected duration. The release profile can be manipulated such as by adjusting features of the amount of agent in the polymeric microsphere.

The ratio of macrocyclic lactone to isoxazoline ranges from about 0.5-3:5. The preferred macrocyclic lactone is moxidectin and the preferred isoxazoline is sarolaner with a moxidectin to sarolaner ratio ranging from about 0.5-1:5, about 0.6-1:5, about 0.63-1:5, about 0.7-1:5, about 0.75-1:5, about 0-8-1:5, about 0.85-1:5, about 0.9-1:5, about 0.95-1:5, about 0.96-1:5 and about 1:5. The preferred moxidectin to sarolaner ratio ranges from about 0.63-1:5, about 0.75-1:5, about 0.95-1:5 and about 1:5. The more preferred ratio of moxidectin to sarolaner is about 0.63:5, 0.76:5 or 0.96:5.

The amount of the isoxazoline (preferred is sarolaner) ranges from about 44% to about 57% and the amount of the macrocyclic lactone (preferred is moxidectin) from about 4% to about 10%. Total combined drug load (isoxazoline+macrocyclic lactone) ranges from about 54% to about 62%; about 57% to about 62% and about 58% to about 60%. A preferred total combined drug load is about 58%, 59% or 60%. Non-limiting examples of total combined drug load for moxidectin (M) and sarolaner(S) include: 6.4% (M) and 53.6% (S), 8.3% (M) and 51.7% (S), 10% (M) and 50% (S); 9.5% M and 50% (S), 5.9% (M) and 49.1% (S); 7.6% (M) and 52.4% (S), 9.2% (M) and 45.8% (S), 8.3% (M) and 51.7% (S), and 9% (M) and 50% (S).

Equally important to controlling the biodegradation of the polymer and the extended-release profile of the microsphere is the relative average particle size of the microsphere. The average size (diameter) of the microsphere ranges from about 20-40 μm, about 24-40 μm, about 24-38 μm, about 24-36 μm, about 25-35 μm, about 26-34 μm and about 26-32 μm, and are polydisperse. A preferred particles size for the microsphere is about 26-34 μm or 26-32 μm.

The extended-release polymeric microsphere composition can be formulated with polymers as a direct injectable depot-like composition. A depot composition is one that can be injected into a desired location in an animal's body to form an implant, which provides for controlled, sustained release of the active agent. More particularly, the present invention pertains to depot compositions comprising a PLGA microsphere containing an isoxazoline (preferably sarolaner) and a macrocylic lactone (preferably moxidectin), and optionally an antioxidant, and at least one pharmaceutically acceptable excipient. Once injected into the animal, the pharmaceutically acceptable excipients of the injected depot diffuse through the tissue while the active agent, which is matrixed with the polymer microsphere is retained in the locally deposited injection site which slowly degrades over time thereby releasing the active agents.

Processes for preparing polymeric microspheres can be prepared by a number of different techniques including phase separation, solvent evaporation, and spray drying. Solvent evaporation techniques can be used to form microspheres. These techniques generally involve dissolving the polymer in a water immiscible organic solvent which contains either dissolved or dispersed active agents (i.e., isoxazoline and macrocyclic lactone). The polymer/active agent solution is then added to an agitated continuous phase which is usually aqueous. Emulsifiers can be included in the aqueous phase to stabilize the oil-in-water emulsion. An emulsion is formed by adding this suspension or solution to a vigorously stirred water (often containing a surface active agent, for example, polyethylene glycol or polyvinyl alcohol, to stabilize the emulsion). The organic solvent is evaporated while continuing to stir. Evaporation results in precipitation of the polymer, forming microspheres containing the active agents. Solvent(s) can be removed from the microspheres in a single step, under reduced pressure as described in U.S. Pat. No. 3,691,090; or by application of heat, as described in U.S. Pat. No. 3,891,570. A two-step technique is described in U.S. Pat. No. 4,389,330. Freeze drying has also been used to remove the solvent from microspheres, as reported by Sato, et al, in “Porous Biodegradable Microspheres for Controlled Drug Delivery. Assessment of Processing Conditions and Solvent Removal Techniques,” Pharmaceutical Research 5, 21-30 (1988).

Phase separation techniques can also be used to form microspheres. These techniques involve the formation of a water-in-oil emulsion or oil-in-water emulsion. The polymer is precipitated from the continuous phase onto the active agent by a change in temperature, pH, ionic strength or the addition of precipitants. For example, U.S. Pat. No. 4,675,800, describes the formation of poly (lactic-co-glycolic) acid microspheres containing active proteins. The protein is first dissolved in the aqueous phase of a water-in-oil emulsion or dispersed as a solid in the polymer phase. Polymer is then precipitated around the aqueous droplets or drug particles by addition of a non-solvent for the biopolymer such as silicone oil.

Spray drying techniques can also be employed to prepare the microspheres. For example, at least one biopolymer is mixed with the active agent together in a solvent, the solvent is evaporated by spraying the solution, leaving polymeric droplets containing the active agent. Spray drying is reviewed in detail by K. Masters in “Spray Drying Handbook” (John Wiley k Sons, New York 1984).

Typical solvents are organic solvents, which can be water miscible or water immiscible. Non-limiting examples of organic solvents include: methylene chloride, chloroform, carbon tetrachloride, dicholorethane, ethyl acetate, methyl acetate, acetone, tetrahydrofuran, N-methylpyrrolidone, N-pyrrolidone, polyvinylpyrrolidone, 2-pyrrolidone, polyvinyl alcohol, cyclohexane, alcohols (e.g., methanol ethanol, n-propanol, isobutyl alcohol, cyclopentyl alcohol, benzyl alcohol, and the like).

The dual active PLGA microspheres of the instant invention can be prepared using a phase separation technique. A dispersed phase containing a solvent (e.g., methylene chloride), PLGA (75:25), sarolaner and moxidectin, and optionally, an antioxidant, are mixed via a homogenizer with the addition of a continuous phase solvent (e.g., mixture of water and polyvinyl alcohol) to form the microspheres. The continuous and dispersed phases are pumped into the homogenizer with the aid of separate peristatic pumps. The microspheres and solvent are passed into a separate vessel containing water. The microspheres are collected using a diafiltration process using hollow fiber filters and the excess water and solvent mixture are discarded. Once prepared, the microspheres are suspended in a mannitol solution, that may optionally contain polysorbate 20 and/or sodium carboxymethyl cellulose, lyophilized and stored to be reconstituted for later use with a diluent (e.g., water and optionally polysorbate 20 and/or sodium carboxymethyl cellulose) at the time to be administered to the animal. In one non-limiting example, the microspheres are suspended in a mannitol and polysorbate 20 solution, lyophilized, and stored to be reconstituted for later use with a diluent (e.g., water and sodium carboxymethyl cellulose) at the time to be administered to the animal. In another non-limiting example, the microspheres are suspended in a mannitol and sodium carboxymethyl cellulose solution, lyophilized, and stored to be reconstituted for later use with a diluent (e.g., water and polysorbate 20) at the time to be administered to the animal. In another non-limiting example, the microspheres are suspended in a mannitol, sodium carboxymethyl cellulose and polysorbate 20 solution, lyophilized, and stored to be reconstituted for later use with water at the time to be administered to the animal. The reconstituting solution can also contain an antioxidant, for example, butylated hydroxytoluene (BHT) or butylated hydroxyanisole (BHA).

In some instances, microspheres containing only an isoxazoline (e.g., sarolaner, fluralaner, lotilaner, and the like) and microspheres containing only a macrocyclic lactone (e.g., moxidectin, ivermectin, milbemycin, and the like) can be prepared separately and then admixed together with mannitol, and optionally sodium carboxymethyl cellulose and/or polysorbate 20, lyophilized and then reconstituted with a solution containing water and optionally, sodium carboxymethyl cellulose and/or polysorbate 20; the reconstituting solution can also contain an antioxidant (e.g., BHT or BHA).

For the extended-release injectable compositions of the instant invention, typical pharmaceutically acceptable excipients include, but are not limited to: water, ethanol, propylene glycol, polyethylene glycols (PEG): PEG 200, PEG 300, and PEG 400; diethylene glycol ethyl ether, isopropylidene glycerol, dimethyl isosorbide, propylene carbonate, glycerol, methylethylketone (MEK), dimethylsulfoxide (DMSO), dipropyleneglycol methyl ether, ethyl lactate, dimethylformamide, N, N-diethyl-m-toluamide, dimethylacetamide, ethylacetamide, caprolactam, decylmethylsulfoxide, triacetin, solketal, propylene carbonate, ethyl lactate, and mixtures thereof. Pharmaceutically acceptable excipients also include: poly (ethylene glycol), polyvinylpyrrolidone, poly (vinyl alcohol) (PVA), cellulose (methyl cellulose, ethyl cellulose, hydroxypropyl cellulose, hydroxypropylmethyl cellulose, hydroxybutyl methyl cellulose, sodium carboxymethyl cellulose, cellulose acetate, cellulose propionate, cellulose acetate butyrate, cellulose acetate phthalate, carboxylethyl cellulose, cellulose triacetate, cellulose sulphate sodium salt), tristearates, polysorbate-type nonionic surfactants (e.g., polysorbate 20 (Tween® 20), polysorbate 60 (Tween® 60, polysorbate 80 (Tween® 80), sugars (e.g., sucrose, lactose, trehalose, and the like), sugar alcohols (e.g., mannitol, sorbitol, and the like), dextran, fatty acids (e.g., decanoic acid, lauric acid, myristic acid, palmitic acid, stearic acid, and the like), fatty alcohols (e.g., lauryl alcohol, myristyl alcohol, cetylic alcohol, stearyl alcohol, and the like), sodium cholate, sodium deoxycholate, sodium glycolate, sodium taurocholate, sodium taurodesoxycholate, lecithin, isotonic modifiers (e.g., sodium chloride, calcium chloride, and the like); preservatives (e.g., citric acid, benzoic acid, ascorbic acid, benzalkonium chloride, benzy alcohol, chlorobutanol, sodium bisulfite, sodium bisulfate, sodium thiosulfate, thimerosal, methylparaben, ethylparaben, propylparaben, and the like), buffers (e.g., phosphate, tromethamine and the like), antioxidants (e.g., butylated hydroxytoluene (BHT), butylated hydroxyanisole (BHA) and the like), rheology modifiers (e.g., xanthan gum, sodium carboxy methyl cellulose, hydroxy propyl methyl cellulose, polyvinyl pyrrolidone, hydroxy ethyl cellulose, hydroxy propyl cellulose, sodium alginate and arginine), and mixtures thereof. Preferred pharmaceutically acceptable excipients include water, polysorbate 20, mannitol, sodium carboxymethyl cellulose, butylated hydroxytoluene and mixtures thereof.

In the present invention, the extended-release injectable PLGA microsphere comprises sarolaner and moxidectin and the composition further comprises at least one pharmaceutically acceptable excipient.

The amount of isoxazoline and macrocyclic lactone compounds are easily determined by a skilled artisan and further depend on the dose amount and dose volume of the final composition. Representative amounts of a pharmaceutically effective amount of an isoxazoline and macrocyclic lactone in the reconstituted finished drug product composition ranges are based upon band presentations which depend on the weight of the animal. Product by weight bands typically include animals ranging from about 2-4 kg, 4.1-8 kg, 8.1-15 kg, 15.1-30 kg and 30.1-60 kg. The concentration of the isoxazoline in the reconstituted composition that is readily administered to the animal ranges from about 20 to 150 mg/ml and about 0.4 to 8 mg/mL for the macrocyclic lactone. The preferred isoxazoline is sarolaner and the preferred macrocyclic lactone is moxidectin. Approximate volumes to be injected into an animal, depending on animal weight, ranges from about 0.25mL to about 3mL. The combined sarolaner and moxidectin drug load in the microsphere ranges from about 54-62% with a moxidectin to sarolaner ratio of about 0.63-1:5.

In dual-active microsphere dissolution assays, it was found that the microspheres dissolve in an aqueous solution over a period of about 24 hours; similar to microspheres containing sarolaner only. In contrast, microspheres containing only moxidectin did not dissolve over several days in the same aqueous media. This finding was unexpected and certainly played a critical role in drug product release dissolution assays and overall optimization of the dual active microsphere.

### Method of Use

The present invention further comprises methods for treating a parasitic infestation in an animal, preferably a companion animal having or being susceptible to infestation, by administering to the animal in need thereof, a therapeutically effective amount of the extended-release polymeric microsphere composition of the invention.

The composition of the present invention is useful as an ectoparasiticide and endoparasiticide that can be used in the fields of veterinary medicine and the maintenance of public health. Non-limiting examples of ectoparasites include: ticks (e.g., Ixodes spp., (e.g., I. ricinus, I. hexagonus), Rhipicephalus spp., (e.g., R. sanguineus), Boophilus spp., Amblyomma spp. (e.g., A. maculatum, A. triste, A. parvum, A. cajennense, A. ovale, A. oblongoguttatum, A. aureolatum, A. cajennense), Hyalomma spp., Haemaphysalis spp., Dermacentor spp. (e.g., D. variabilis, D. andersoni, D. marginatus), Ornithodorus spp., and the like); mites (e.g., Dermanyssus spp., Sarcoptes spp., (e.g., S. scabiei), Psoroptes spp., (e.g., P/bovis), Otodectes spp., Chorioptes spp., Demodex spp., (e.g., D. folliculorum, D. canis, and D. brevis) and the like); chewing and sucking lice (e.g., Damalinia spp., Linognathus spp., Cheyletiella spp., Haematopinus spp., Solenoptes spp., Trichodectes spp., Felicola spp., and the like); fleas (e.g., Siphonaptera spp., Ctenocephalides spp., and the like); biting flies, midges, and mosquitoes (e.g., Tabanidae spp., Haematobia spp., Musca spp., Stomoxys spp., Dematobia spp., Cochliomyia spp., Simuliidae spp., Ceratopogonidae spp., Psychodidae spp., Aedes spp., Culex spp., Anopheles spp., and the like); bed bugs (e.g., insects within the genus Cimex and family Cimicidae); and grubs (e.g., Hypoderma bovis, H. lineatum). Some non-limiting examples of endoparasites include: gastrointestinal roundworms (e.g., Ostertagia ostertagi, O. lyrata, Haemonchus placei, H. similis, H. contortus, Toxocara canis, T. leonina, T. cati, Trichostrongylus axei, T. colubriformis, T. longispicularis, Cooperia oncophora, C. pectinata, C. punctata, C. surnabada (syn. mcmasteri), C. spatula, Ascaris suum, Hyostrongylus rubidus, Bunostomum phlebotomum, Capillaria bovis, B. trigonocephalum, Strongyloides papillosus, S. ransomi, Oesophagostomum radiatum, O. dentatum, O. columbianum, O. quadrispinulatum, Trichuris spp., and the like); hookworms (e.g., Ancylostoma caninum, A. tubaeforme, A. braziliense, A. ceylanicum, Uncinaria stenocephala); lungworms (e.g., Dictyocaulus viviparus and Metastrongylus spp); eyeworms (e.g., Thelazia spp.); parasitic stage grubs (e.g., Hypoderma bovis, H. lineatum, Dermatobia hominis); kidneyworms (e.g., Stephanurus dentatus); screw worm (e.g., Cochliomyia hominivorax (larvae); filarial nematodes (Brugia spp. (e.g., B. malayi, B. pahangi, B. timori, and the like), Wuchereria spp. (e.g., W. bancrofti, and the like), Dirofilaria spp. (D. immitis, D. repens, D. ursi, D. tenuis, D. spectans, D. lutrae, and the like), Dipetalonema spp. (e.g., D reconditum, D. repens, and the like), Onchocerca spp. (e.g., O. gibsoni, O. gutturosa, O. volvulus, and the like), Elaeophora spp. (e.g., E. bohmi, E. elaphi, E. poeli, E. sagitta, E. schneideri, and the like), Mansonella spp. (e.g., M. ozzardi, M. perstans, and the like), and Loa spp. (e.g., L. loa).

The compositions of the invention can be administered in a way appropriate to the specific use envisaged, the particular host animal and weight of host animal being treated, the parasite or parasites involved, degree of infestation, etc., according to standard veterinary practice. The veterinary practitioner, or one skilled in the art, will be able to determine the dosage suitable for the particular animal which may vary with the species, age, weight, and response. The average doses are exemplary of the average case. Accordingly, higher or lower dosage ranges may be warranted, depending upon the above factors, and are within the scope of this invention.

The method of treating an animal with a parasitic infestation comprises the administration of the extended-release polymeric microsphere composition comprising a therapeutically effective amount of an isoxazoline (preferably sarolaner) and a maxcrocyclic lactone (preferably moxidectin). Administration is contemplated as a long-term injectable composition. In particular, injectable administration is considered to be subcutaneous injection. The composition can be administered to the animal in need thereof, by administering an effective amount of the polymeric composition thereof to the animal at least once every 3-months, 4-months, 5-months, 6-months, 7-months, 8-months, 9-months, 10-months, 11-months, or 12-months. The preferred dosing administration is contemplated to be at least once every 6 or 12 months.

Moreover, for animal administration, it will be understood that preparations should meet sterility, pyrogenicity, general safety and purity standards as required by local regulatory agencies.

The present invention also relates to a method of administering the PLGA (or PLA) microsphere composition to an animal in good health comprising the administration of the microsphere composition to said animal to reduce or eliminate the parasitic infection.

### EXAMPLES

### Composition Examples

The following dual active compositions shown in Table 1 were prepared.

TABLE 1
Dual Active Micrsophere Drug Loading

| Composition | | | Total Drug | |
|---|---|---|---|---|
| # | % Sarolaner | % Moxidectin | Load % | Ratio S:M |
| 1 | 45 | 9 | 54 | 5:1 |
| 2 | 40 | 16 | 56 | 5:2 |
| 3 | 35 | 21 | 56 | 5:3 |
| 4 | 51.6 | 10.4 | 62 | 5:1 |

For composition 1, the dispersed phase was prepared by dissolving 13.8 g of PLGA (75:25) polymer in 120 g of dichloromethane (DCM). To this PLGA/DCM mixture was dissolved 2.7 g moxidectin and 13.5 g sarolaner with continuous mixing for about 10 minutes to prepare about 30 g of dual active microspheres. The continuous phase consists of a mixture of water and polyvinyl alcohol (PVA). The amount of PVA in water ranges from about 0.2 to 3%. Both the dispersed and continuous phases were pumped into a homogenizer at a specific ratio wherein an emulsion was rapidly created at the intersection of the two fluids in the homogenizer. As a result of the flow rates, solvent rapidly diffuses from the emulsion thereby creating the dual active microspheres. The solvent and microspheres were expelled from the homogenizer and were collected in filtered deionized water. The suspended microspheres were filtered via hollow fiber filters to remove the DCM and PVA. The microspheres were then collected from the aqueous suspension and mixed with an aqueous solution containing mannitol, ranging from about 30 to 70 mg/mL mannitol. The microsphere/mannitol suspension was added to glass vials. Depending on dose/volume to be administered for weighted dog range, concentrations of sarolaner, moxidectin, PLGA and mannitol concentrations vary as shown in Table 2,

TABLE 2
Pre-lyophilized Drug Concentrations

| Sarolaner | Moxidectin | PLGA |
|---|---|---|
| (mg) | (mg) | (mg) |
| 20 | 4 | 18 |
| 40 | 8 | 36 |
| 75 | 15 | 68 |
| 150 | 30 | 136 |
| 300 | 60 | 272 |

The vials containing the aqueous suspension of microspheres and mannitol were lyophilized. The lyophilized product is then resuspended from a diluent containing a mixture of sodium carboxymethyl cellulose, polysorbate 20 and water for injection.
Upon reconstitution, the final concentration of sarolaner across the dog weight range (lowest to highest) is about 20, 40, 75, 75, and 100 mg/ml; and the respective moxidectin concentrations are about 4, 8, 15, 15 and 20 mg/ml (Table 3). Overall, the sarolaner: moxidectin concentration ratio for the finished product was 5:1.

TABLE 3
Reconstituted Microsphere Concentrations and Volumes

| Sarolaner | Moxidectin | Injection Volume |
|---|---|---|
| (mg/mL) | (mg/mL) | (mL) |
| 20 | 4 | 1 |
| 40 | 8 | 1 |
| 75 | 15 | 1 |
| 75 | 15 | 2 |
| 100 | 20 | 3 |

### Biological

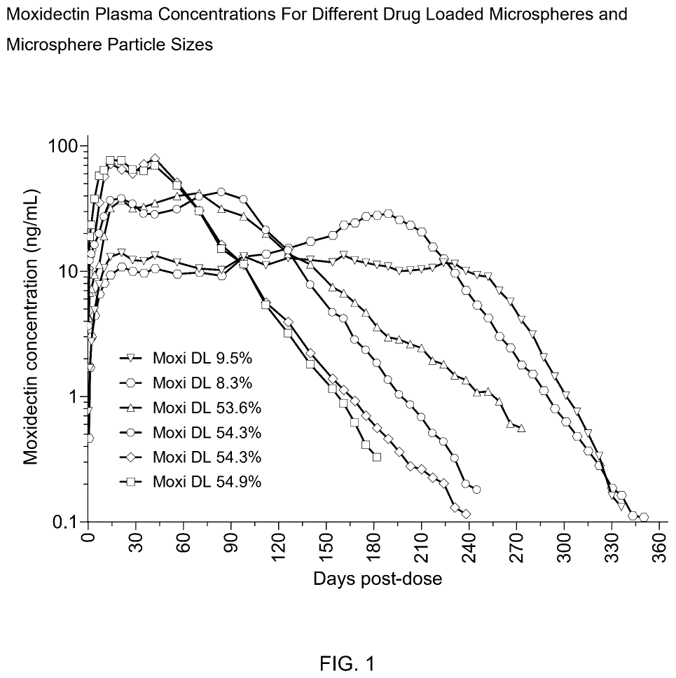

In a study, the pharmacokinetics of sarolaner and moxidectin were assessed following subcutaneous administration of microspheres that contained moxidectin or microspheres that contained both moxidectin and sarolaner. Target doses of moxidectin were 2 mg/kg and target dose of sarolaner was 10 mg/kg. Dual active microsphere suspension doses comprising 16 mg/ml moxidectin and 77 mg/mL sarolaner or single active 16 mg/mL moxidectin microspheres were prepared using different percent drug loading and particle sized microspheres. For the moxidectin only microspheres, drug loading was about 54%. For the dual (moxidectin and sarolaner) active microspheres, total combined drug load was about 60% (about 50% sarolaner and about 10% moxidectin). Particle size for all microspheres ranged from about 32.5 μm to about 51 μm. Male beagle dogs (4/group) weighing >6 kg and >than 12-months of age were used in the study. Animals were administered 0.125 mL/kg by subcutaneous injection. Plasma from blood samples was obtained 4 hours prior to dosing, 6-hours post-dose, and then on Days 1, 2, 4, 7, 10, 14, 21, 28, 35, 42, 56, 70, 84, 98, 112, 126, 140, 154, 161, 168, 175, 182, 189, 196, 203, 210, 217, 224, 231, 238, 245, 252, 259, 266, 273, 280, 287, 294, 301, 308, 315, 322, 330, 336, 343, 350, 357 and 364 days post-dose at approximately the same time of dosing on Day 0. PLGA microsphere drug loading (DL) was 53.6% moxidectin (Lot #1); 54.3% moxidectin (Lot 2; designated as -o- in FIG. 1); 54.3% moxidectin (Lot #3); 54.9% moxidectin (Lot #4); 9.5% moxidectin and 49.9% sarolaner (Lot #5); and 8.3% moxidectin and 43.4% sarolaner (Lot #6). Dual active PLGA microsphere characteristics are shown in Table 4, wherein the L: G ratio refers to the lactide: glycolide ratio of the PLGA. The inherent viscosity (IV) of the PLGA polymer was measured at 25° C.; 0.5% CHCl₃ for Lots 1, 5 and 6.

TABLE 4
PLGA Polymer Characteristics

| | | | Particle | |
|---|---|---|---|---|
| | | Drug | Size | IV |
| Lot # | L:G Ratio | Load (%) | (μm) | (dL/g) |
| 1 | 75:25 | 53.6 | 51.5 | 0.30 |
| 2 | 75:25 | 54.3 | 35.2 | 0.39 |
| 3 | 50:50 | 54.3 | 42.0 | 0.42 |
| 4 | 65:35 | 54.9 | 34.5 | 0.39 |
| 5 | 75:25 | 59.4\hat{ } | 32.5 | 0.39 |
| 6 | 75:25 | 51.7\hat{ } | 33.1 | 0.39 |

\hat{ } - Combination of sarolaner and moxidectin

The mean plasma moxidectin concentrations for dogs in each of the treatment groups is shown in FIG. 1. The minimal effective concentration contemplated for round worm is about 10 ng/ml moxidectin.

As can be observed in FIG. 1, PLGA microspheres with a L:G ratio of 50:50 (Lot #3) or 65:35 (Lot #4) with similar moxidectin drug load (Moxi DL) (54.6%) with slightly different particle sizes (42 μm and 34.5 μm) and similar IV's (0.42 dL/g and 0.39 dL/g) provided quick release kinetics for moxidectin resulting in a higher Cmax between about 14 and 56 days but fell below the 10 ng/ml lowest efficacious dose for round worm at around 98 days. The release from the larger microspheres (Lot #3) was slightly slower than the smaller microspheres (Lot #4). The PLGA microspheres with an 75% poly (D,L-lactide) and 25% polyglycolide (Lots 1 and 2) with about 54% moxidectin drug loading, similar IV's (0.39 dL/g) and dissimilar particle sizes had similar Cmax values which released moxidectin at a slower rate than the microspheres from Lots #3 and #4; and showed that the larger microsphere actually released moxidectin at a slower rate. Regardless, both Lots #1 and #2 moxidectin concentrations fell below the 10 ng/ml value needed for round worm efficacy at around 126 days. Surprisingly, lower amounts of moxidectin combined with sarolaner in similarly sized microspheres (32.5 μm and 33.1 μm) with the same IV (0.39 dL/g) released moxidectin at a much slower rate than the other microspheres containing moxidectin alone. An increase in moxidectin plasma levels was observed at about 154 to 196 days for the lower drug loaded microsphere (Lot 6, 51.7%) and yet both combined drug micrsopheres maintained moxidectin concentrations at or above the 10 ng/ml round worm concentration for at least 238 days, after which both lots entered an elimination phase of consistently decreasing moxidectin plasma levels.

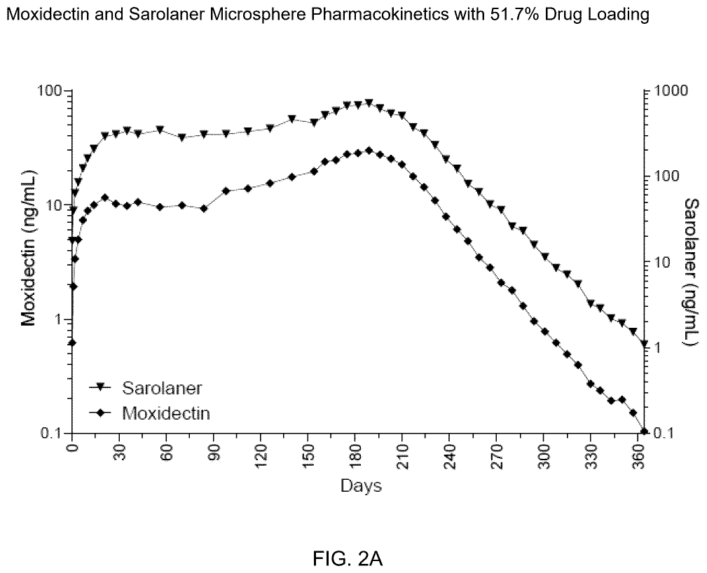

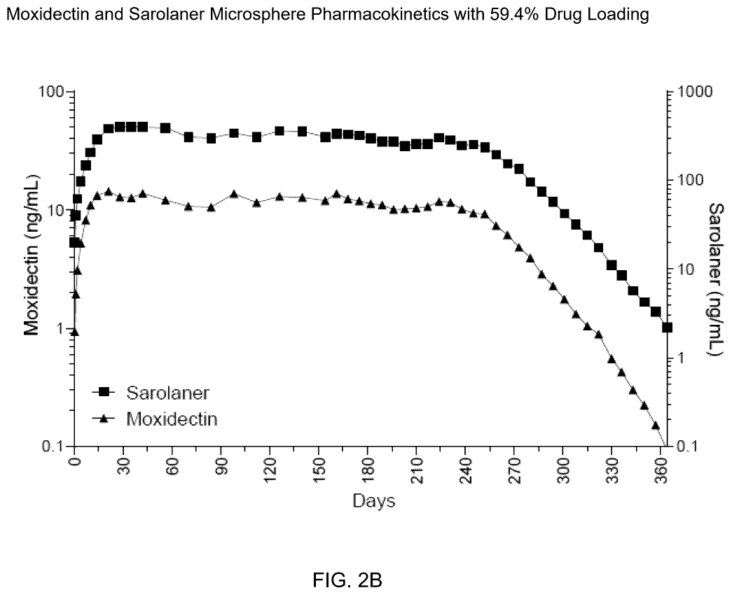

Different plots of the dual active microspheres are shown in FIGS. 2a and 2b which include both the concentrations of moxidectin and sarolaner. The sarolaner in the lower drug loaded (51.7% (Lot 6)) versus higher drug loaded (59.4% (Lot 5)) and slightly larger micropsheres (33.1 vs 32.5) was released more slowly over time. Overall, drug load (moxidectin and sarolaner), L: G ratio, particle size and IV are key drivers for ensuring efficacious drug concentrations for the dosing duration.

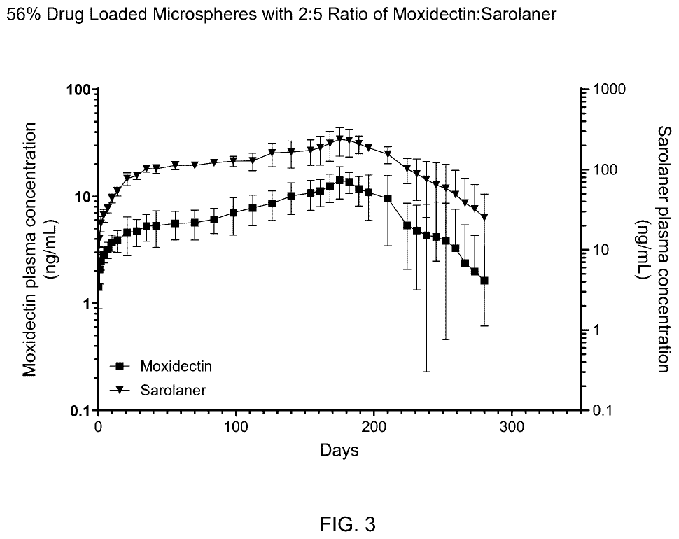

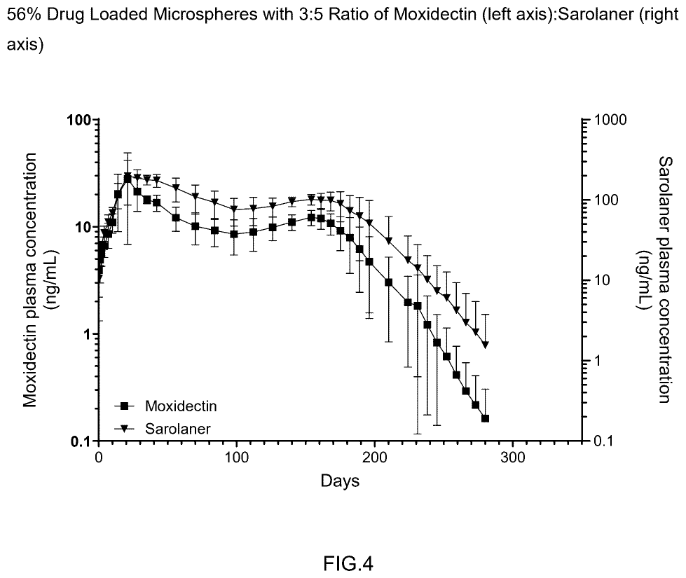

In a separate study, drug load was 56% (FIG. 3) with a moxidectin: sarolaner ratio of 2:5 (16% moxidectin and 40% sarolaner) in microspheres with a particle size of 34 μm and an IV of 0.39 dL/g that showed lower release kinetics for moxidectin wherein moxidectin did not reach the 10 ng/ml concentration until about Day 140 and fell below the 10 ng/ml concentration at around Day 210 while achieving a Cmax of about 15 ng/ml at around Day 170 while sarolaner achieved the 100 ng/ml concentration around Day 10 and fell below the 100 ng/ml concentration around Day 220 while achieving a Cmax of about 160 ng/ml at around Day 170. Subsequently, a separate study with a 56% drug load (FIG. 4) but with a 3:5 ratio of moxidectin: sarolaner (21% moxidectin and 35% sarolaner) with a particle size of 35 μm and an IV of 0.39 dL/g showed much more rapid release of moxidectin which achieved the 10 ng/ml range at around Day 10 and falling below 10 ng/L at around Day 200 while sarolaner achieved the 100 ng/ml concentration around Day 10 and falling below 100 ng/mL at around Day 40.

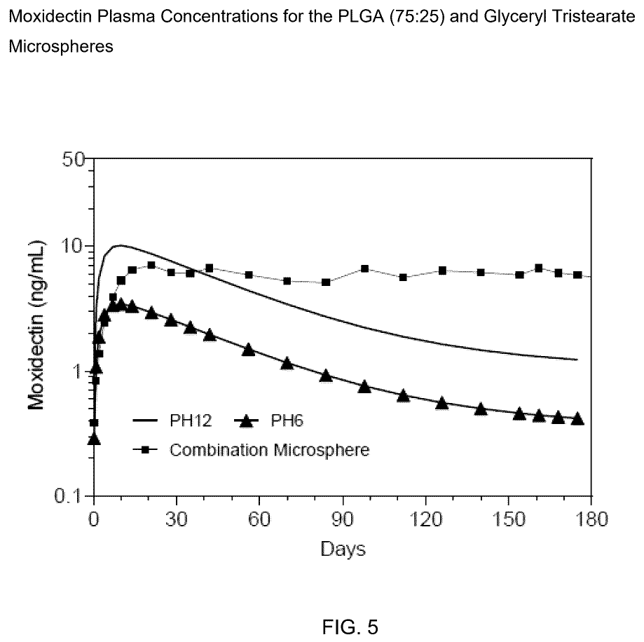

In comparison, moxidectin microspheres prepared from a high melting fat that is glyceryl tristearate and suspended in an injectable formulation (Proheart 6 (PH6) and Proheart 12 (PH12)) were compared to the dual-active PLGA (75:25, 32.5 μm, 0.39 dL/g) microspheres. The 6-month and the 12-month injectable products are similar, as described herein, with the primary difference being a 3× increase in the mass/number of micrsopheres per mL for injection versus the 6-month formulation. Animals received a 1 mg/kg dose of the dual active PLGA microsphere composition, a 0.5 mg/kg dose of the PH12 formulation and 0.17 mg/kg of the PH6 formulation, according to Proheart dosing instructions. As can be seen in FIG. 5, the release of moxidectin is greatly extended over time compared to the Proheart concentrations with a similar Cmax concentration. The PLGA microsphere provided longer extended-release duration of moxidectin than did either of the Proheart microsphere formulations.

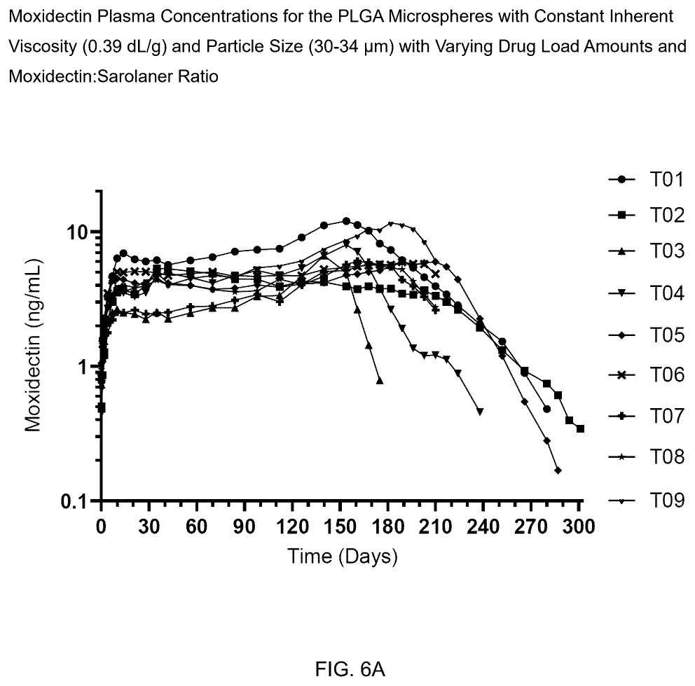

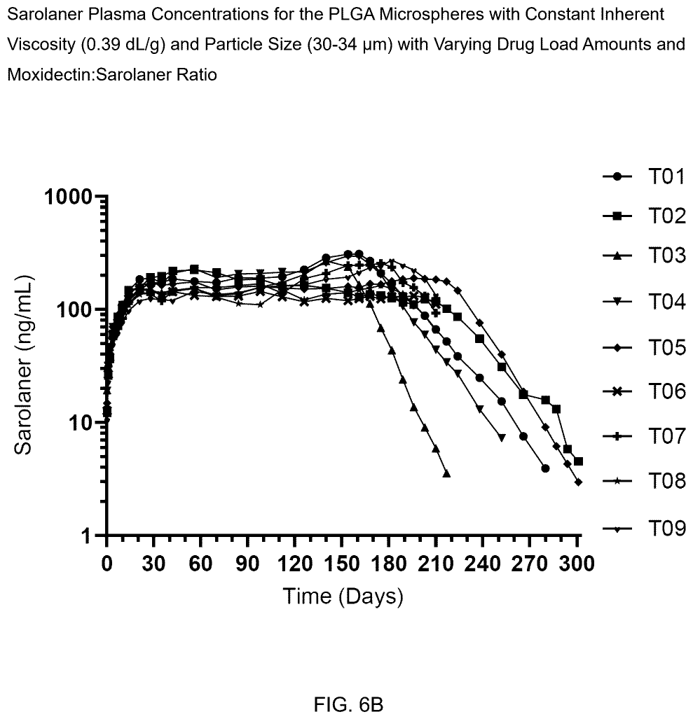

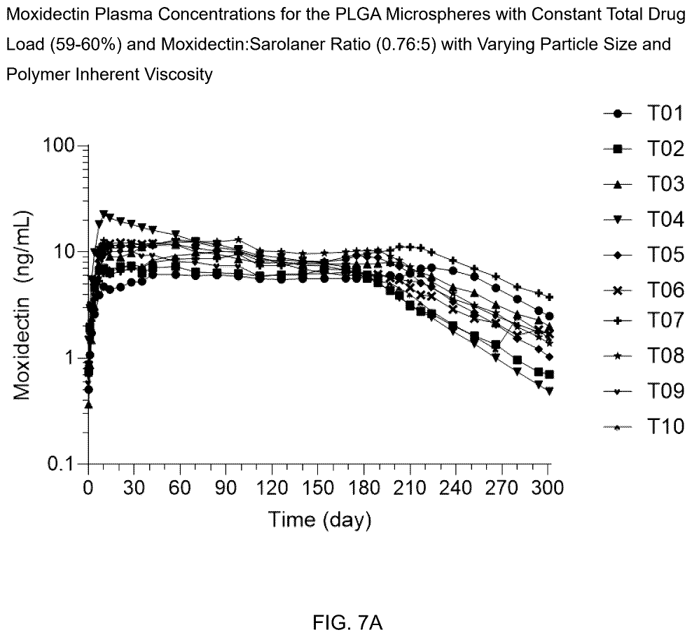

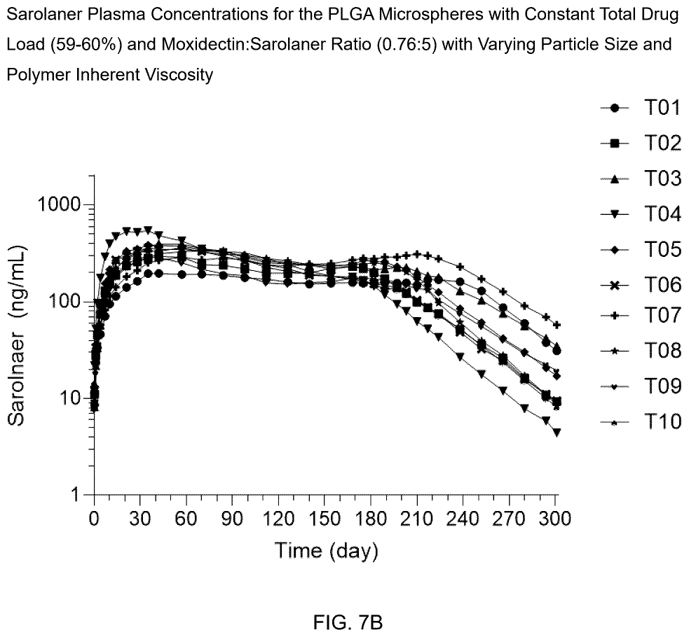

In two additional studies, 4 dogs per treatment group received subcutaneous injections (0.067 mL/kg) of the extended-release PLGA (75% poly (D,L-lactide) and 25% polyglycolide) compositions. Dogs received a 5 mg/kg dose of sarolaner. In one study, inherent viscosity (iv; 0.39 dL/g) and microsphere particle size (30-34 μm) were kept relatively constant while combined total drug load (49%-59%) and moxidectin: sarolaner ratio (M: S; 0.58-0.96:5) were varied (Table 5 (A) and FIGS. 6a and 6b); and then in the other study, combined total drug load (59-60%) and moxidectin: sarolaner ratio (0.76:5) were kept relatively constant [moxidectin 7.5-8% and sarolaner 50-52%] while inherent viscosity (0.26-0.39 dL/g) and microsphere particle size (21-33 μm) were varied (Table 5 (B) and FIGS. 7a and 7b).

TABLE 5
Modified Microsphere Characteristics
A
inherent viscosity (0.39 dL/g)
and microsphere particle size (30-34

| | Total Combined | |
|---|---|---|
| | Drug Load % | Ratio (M:S) |
| T01 | 49 | 0.95:5 |
| T02 | 59 | 0.59:5 |
| T03 | 49 | 0.58:5 |
| T04 | 49 | 0.76:5 |
| T05 | 59 | 0.76:5 |
| T06 | 59 | 0.96:5 |
| T07 | 53 | 0.58:5 |
| T08 | 54 | 0.76:5 |
| T09 | 54 | 0.96:5 |

B
Total combined drug load (59-60%)
and moxidectin:sarolaner ratio (0.76:5)

| | | particle size |
|---|---|---|
| | iv (dL/g) | (μm) |
| T01 | 0.39 | 26 |
| T02 | 0.26 | 27 |
| T03 | 0.31 | 26 |
| T04 | 0.26 | 20 |
| T05 | 0.31 | 28 |
| T06 | 0.31 | 21 |
| T07 | 0.39 | 32 |
| T08 | 0.31 | 31 |
| T09 | 0.26 | 33 |
| T10 | 0.39 | 21 |

The plasma moxidectin (FIGS. 6a and 7a) concentrations and sarolaner concentrations (FIGS. 6b and 7b) were obtained following single subcutaneous doses of the test microsphere compositions of the last two dog studies. Even though sarolaner and moxidectin amounts (and ratio) varied due to drug loading, sarolaner dose was held constant at about 5 mg/kg while moxidectin dose ranged from about 0.6-1.0 mg/kg. The efficacious dose for treating ectoparasites with sarolaner and endoparasites with moxidectin is contemplated to be about 80 ng/ml and 5 ng/ml, respectively. FIGS. 6a and 6b display formulations with consistent intrinsic viscosity (0.39 dL/g) and particle size (30-34 μm) and varied total drug load (near 49%, 54%, 59%) and varied ratio of moxidectin to sarolaner (near 0.6 mg/kg, 0.8 mg/kg, 1 mg/kg). The results support that total drug load is associated with duration of release, with 49% drug load exhibiting release that was less than desired at 180 days, 54% total drug load near 190 days, and 59% total drug load near 210-220 days. A critical metric for the pharmacokinetics of moxidectin is maintaining a consistent concentration across the plateau phase (i.e., about 59% drug load). Lastly, moxidectin concentrations in the 0.6 mg/kg group were lower than the putative 5 ng/ml threshold for endoparasites. FIGS. 7a and 7b show formulations with a constant total drug load (near 59%) and ratio (near 0.8 mg/kg moxidectin) with varied particle size (near 20 μm, 26 μm, 32 μm) and varied inherent viscosity (0.26 dL/g, 0.31 dL/g, 0.39 dL/g). The results demonstrate a positive correlation between polymer inherent viscosity and duration of release, with the 0.31 dL/g and 0.39 dL/g exhibiting acceptable profiles in terms of moxidectin and sarolaner plasma concentration duration. When assessing the consistency of moxidectin across the concentration time profile, particle sizes near 20 μm exhibited unacceptable fluctuations in moxidectin levels. Overall, acceptable plasma pharmacokinetics were observed when the total drug load was above 54%, the particle size was between 26 μm-34 μm, the polymer inherent viscosity was between 0.31 dL/g to 0.39 dL/g and following a 0.76 mg/kg to 1 mg/kg moxidectin dose and a 5 mg/kg sarolaner dose for a moxidectin to sarolaner ratio of about 0.76-1:5.

We claim:

1. An extended-release antiparasitic composition comprising PLGA or PLA polymeric microspheres ranging from about 24 μm to about 40 μm; and wherein the microspheres contain an isoxazoline and a macrocyclic lactone with a total combined microsphere drug load of about 54% to 62%; wherein the isoxazoline is selected from the group consisting of afoxolaner, esafoxolaner, fluralaner, lotilaner, mivorilaner, sarolaner and umifoxolaner and the macrocyclic lactone is selected from the group consisting of abamectin, eprinomectin, selamectin, doramectin, moxidectin and milbemycin oxime; the composition further comprises at least one pharmaceutically acceptable excipient; and wherein extended-release is a duration of at least 6 months.

2. The extended-release antiparasitic composition of claim 1, wherein the polymer is a PLGA polymer that comprises about 50% to about 75% poly (D,L-lactide) and about 50% to about 25% polyglycolide; and wherein the isoxazoline is sarolaner and the macrocyclic lactone is moxidectin.

3. The extended-release antiparasitic composition of claim 2, wherein the PLGA polymer comprises about 75% poly (D,L-lactide) and about 25% polyglycolide.

4. The extended-release antiparasitic composition of claim 3, wherein the inherent viscosity of the PLGA polymer is about 0.28 dL/g to about 0.43 dL/g and the microsphere moxidectin to sarolaner ratio is about 0.63-1:5.

5. The extended-release antiparasitic composition of claim 4, wherein the microspheres have a particle size of about 24 μm to about 40 μm.

6. The extended-release antiparasitic composition of claim 5, wherein the total combined microsphere drug load is about 57% to about 62% and wherein the microspheres have a particle size of about 24 μm to about 36 μm.

7. The extended-release antiparasitic composition of claim 6, wherein the PLGA polymer inherent viscosity is about 0.28 dL/g to about 0.41 dL/g.

8. The extended-release antiparasitic composition of claim 7, wherein the PLGA polymer inherent viscosity is about 0.30 dL/g to about 0.40 dL/g; the microspheres have a particle size of about 25 μm to about 35 μm and the microsphere moxidectin to sarolaner ratio is about 0.75-1:5.

9. The extended-release antiparasitic composition of claim 8, wherein the microspheres further comprise an antioxidant.

10. The extended-release antiparasitic composition of claim 9, wherein the antioxidant is butylated hydroxytoluene.

11. The extended-release antiparasitic composition of claim 1, wherein the polymer is PLGA that is 75% poly (D,L-lactide) and 25% polyglycolide with an inherent viscosity of about 0.30 dL/g to about 0.40 dL/g; the microspheres have a particle size of about 25 μm to about 35 μm; the macrocyclic lactone is moxidectin and the isoxazoline is sarolaner with a total combined microsphere drug load of about 57% to about 62% with a moxidectin to sarolaner ratio of about 0.75-1:5; and wherein the at least one pharmaceutically acceptable excipient is selected from the group consisting of mannitol, polysorbate 20, sodium carboxymethyl cellulose, water and mixtures thereof.

12. The extended-release antiparasitic composition of claim 11, wherein the PLGA polymer inherent viscosity is about 0.31 dL/g to about 0.39 dL/g, the microspheres have a particle size of about 26 μm to about 34 μm and wherein the microspheres further comprise the antioxidant butylated hydroxytoluene.

13. The extended-release antiparasitic composition of claim 12, wherein the composition further comprises butylated hydroxytoluene or butylated hydroxyanisole.

14. The extended-release antiparasitic composition of claim 1, wherein the polymer is PLGA that is 75% poly (D,L-lactide) and 25% polyglycolide with an inherent viscosity of about 0.31 dL/g to about 0.39 dL/g; the macrocyclic lactone is moxidectin and the isoxazoline is sarolaner with a total combined microsphere drug load of about 57% to about 62% with a moxidectin to sarolaner ratio of about 0.75-1:5; the microspheres have a particle size of about 26 μm to about 34 μm and wherein the microspheres further comprise butylated hydroxytoluene.

15. The extended-release antiparasitic composition of claim 14, wherein the at least one pharmaceutically acceptable excipient is selected from the group consisting of mannitol, polysorbate 20, sodium carboxymethyl cellulose, water and mixtures thereof.

16. The extended-release antiparasitic composition of claim 15, wherein the composition further comprises butylated hydroxytoluene or butylated hydroxyanisole.

17. The extended-release antiparasitic composition of claim 1, wherein the polymer is a PLA polymer with an inherent viscosity of about 0.6-1.0 dL/g; the macrocyclic lactone is moxidectin and the isoxazoline is sarolaner with a microsphere moxidectin to sarolaner ratio of about 0.6-1:5; and wherein the microspheres have a particle size of about 25 μm to about 38 μm and wherein the microspheres further comprise butylated hydroxytoluene.

18. The extended-release antiparasitic composition of claim 17, wherein the at least one pharmaceutically acceptable excipient is selected from the group consisting of mannitol, polysorbate 20, sodium carboxymethyl cellulose, water, butylated hydroxytoluene, butylated hydroxyanisole and mixtures thereof.

19. A method of treating or preventing a parasitic infection in a companion animal by administering the extended-release antiparasitic composition of claim 1 to an animal in need thereof.

20. The method of claim 19, wherein the companion animal is canine and feline and the extended-release antiparasitic composition is administered by subcutaneous injection.
